(19) United States
(12) Patent Application Publication
(10) Pub. No.: US 2017/0233124 A1
(43) Pub. Date: Aug. 17, 2017

(54) METHOD AND DEVICE FOR PACKAGING ELASTOMER PARTS

(71) Applicant: APTAR STELMI SAS, Villepinte (FR)

(72) Inventors: Ghislain FOURNIER, La Rochelle (FR); Francois DESSUS, Orsay (FR)

(73) Assignee: APTAR STELMI SAS, Villepinte (FR)

(21) Appl. No.: 15/519,205

(22) PCT Filed: Oct. 12, 2015
(86) PCT No.: PCT/FR2015/052745

§ 371 Date: Apr. 14, 2017

(30) Foreign Application Priority Data
Oct. 17, 2014 (FR) 1460016

(51) Int. Cl. B65B 55/10; B65B 55/16; A61L 2/08; B65D 65/38; B65D 75/38; B65D 81/20; B65B 31/00; A61J 1/00

(52) U.S. Cl. CPC B65B 55/10; B65B 31/00; B65B 55/16; A61J 1/00; B65D 65/38; B65D 75/38; B65D 81/2084; A61L 2/081; A61L 2202/23

(57) ABSTRACT

A packaging method for packaging elastomer parts (1), such as stoppers for pharmaceutical containers, the method comprising the following steps:
packaging parts (1) in a primary bag (10) made of material that is substantially impermeable to gas; and
introducing in said primary bag (10), a nitrogen-based atmosphere, with at least 80% nitrogen; said primary bag (10) being packaged in a secondary bag (20), with a vacuum being applied between said primary bag (10) and said secondary bag (20)

The present invention relates to a packaging method and device for packaging parts made of elastomer, such as stoppers for pharmaceutical containers.

Pharmaceutical containers for containing pharmaceuticals are generally closed, after filling, in leaktight and sterile manner by appropriate stoppers, typically made of elastomer materials, such as rubber. Such stoppers are generally delivered separately from the containers to the user responsible for filling, the stoppers being packaged in appropriate packaging devices. Such ready-to-use stoppers are generally packaged in the ambient atmosphere in a primary bag having good impermeability to air, typically a bag made of polyethylene (PE) or aluminum. The primary bag is heat-sealed without being put under a vacuum. The primary bag may itself be inserted into a secondary bag, generally of the polyethylene (PE) type, or more particularly of the three-layer polyethylene/polyamide/polyethylene (PE/PA/PE) type, that is designed to be put under vacuum.

Such packaging systems present drawbacks.

Thus, the elastomer stoppers tend to stick to one another, and this phenomenon is amplified over time (during storage) and still more after sterilization.

The same problem may occur for other types of elastomer parts, such as syringe pistons or syringe needle-guards, for example.

Documents JP 2003/291917, DE 3 717 916, WO 2011/090942, and EP 0 201 880 describe prior-art devices.

An object of the present invention is to provide a packaging method and device for packaging elastomer parts that do not have the above-mentioned drawbacks.

In particular, an object of the present invention is to guarantee a very little, advantageously no, sticking between the parts contained in the primary bag of the packaging device, even after sterilization.

The present invention thus provides a packaging method for packaging elastomer parts, such as stoppers for pharmaceutical containers, the method comprising the following steps: packaging parts in a primary bag made of material that is substantially impermeable to air; and applying in said primary bag, a nitrogen-based atmosphere, with at least 80% nitrogen; said primary bag being packaged in a secondary bag, with a vacuum being applied between said primary bag and said secondary bag.

The present invention also provides a packaging device for packaging elastomer parts, such as stoppers for pharmaceutical containers, the device comprising a primary bag, made of material that is substantially impermeable to air, receiving said parts, said primary bag containing a nitrogen-based atmosphere, with at least 80% nitrogen, said primary bag being packaged in a secondary bag, with a vacuum being applied between said primary bag and said secondary bag.

Advantageously, the atmosphere applied in said primary bag contains at least 90% nitrogen.

Advantageously, the atmosphere applied in said primary bag contains 100% nitrogen.

Advantageously, the atmosphere applied in said primary bag is constituted substantially of nitrogen and of oxygen.

Advantageously, said primary bag that is substantially impermeable to air comprises polyethylene or aluminum.

Advantageously, said secondary bag comprises a plurality of layers, in particular a three-layer polyethylene/polyamide/polyethylene (PE/PA/PE) structure.

The present invention also provides a packaging method for packaging elastomer parts, such as stoppers for pharmaceutical containers, the method comprising the following steps: packaging parts in a primary bag made of material that is permeable to air; packaging said primary bag in a secondary bag made of material that is substantially impermeable to air; and applying in said secondary bag, a nitrogen-based atmosphere, with at least 80% nitrogen; said secondary bag being packaged in a tertiary bag, with a vacuum being applied between said secondary bag and said tertiary bag.

The present invention also provides a packaging device for packaging elastomer parts, such as stoppers for pharmaceutical containers, the device comprising a primary bag, made of material that is permeable to air, receiving said parts, said primary bag being packaged in a secondary bag made of material that is substantially impermeable to air, said primary bag and said secondary bag containing a nitrogen-based atmosphere, with at least 80% nitrogen, said secondary bag being packaged in a tertiary bag, with a vacuum being applied between said secondary bag and said tertiary bag.

Advantageously, the atmosphere applied in said secondary bag contains at least 90% nitrogen.

Advantageously, the atmosphere applied in said secondary bag contains 100% nitrogen.

Advantageously, the atmosphere applied in said secondary bag is constituted substantially of nitrogen and of oxygen.

Advantageously, said primary bag that is permeable to air comprises polyethylene, in particular Tyvek®.

Advantageously, said secondary bag includes a plurality of layers, in particular a four-layer structure with an inner layer made of polyethylene terephthalate, a layer made of aluminum, a layer made of polyamide, and an outer layer made of polyethylene.

Advantageously, said tertiary bag comprises polyethylene.

Advantageously, after applying the nitrogen-based atmosphere in the primary bag or in the secondary bag, said parts are subjected to gamma sterilization.

These characteristics and advantages and others of the present invention appear more clearly from the following detailed description, given by way of non-limiting example, and with reference to the accompanying drawing, in which:

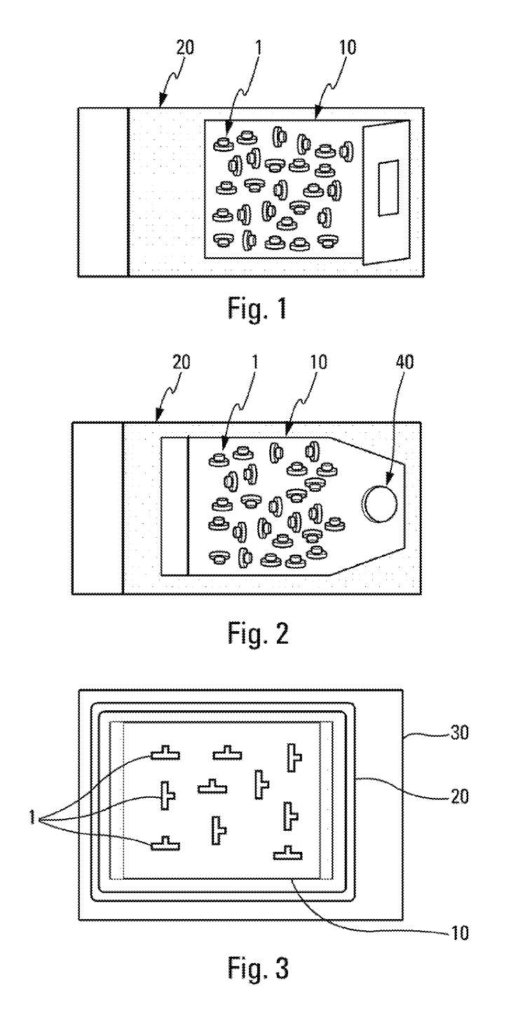

FIG. 1 is a diagrammatic view of a packaging device for packaging elastomer parts comprising stoppers, constituting a first embodiment;

FIG. 2 is a diagrammatic view of a packaging device for packaging elastomer parts comprising stoppers, constituting a second embodiment;

FIG. 3 is a diagrammatic view of a packaging device for packaging elastomer parts comprising stoppers, constituting a third embodiment;

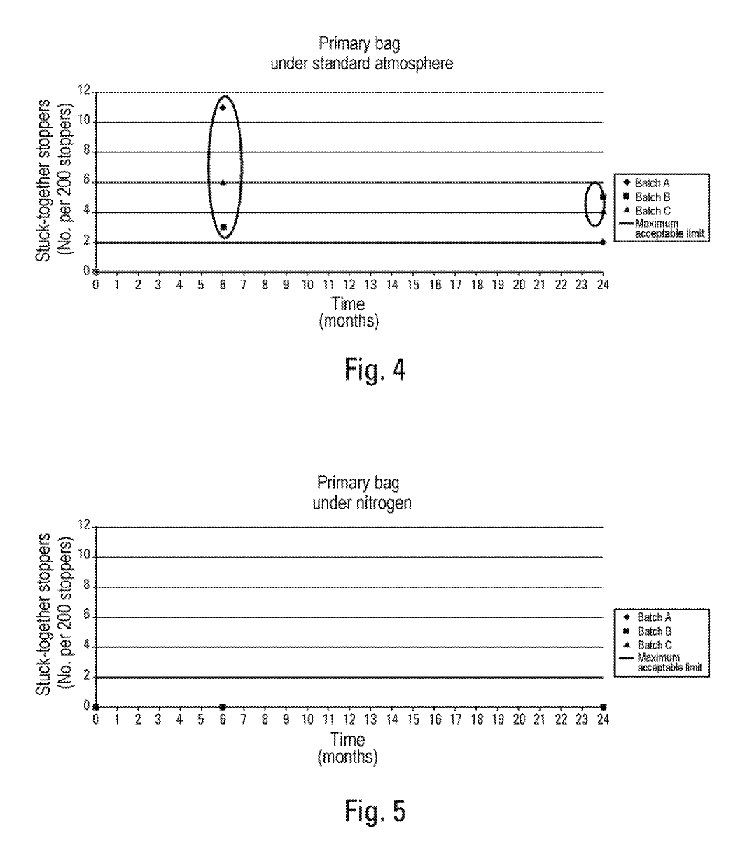

FIG. 4 is a graph showing the performance of a packaging device of the prior art; and

FIG. 5 is a graph showing the performance of a packaging device of the invention,

FIGS. 1 to 3 are diagrammatic, and naturally neither the shape of the parts nor the shape of the various bags is representative of the real shapes or to scale. In particular, the parts may be of any number and of any shape, and the bags may also be of any shape and size.

In conventional manner, elastomer parts 1, such as stoppers for pharmaceutical containers, are packaged in a packaging device that includes a primary bag 10 that receives said parts directly.

Advantageously, the packaging device further includes a secondary bag 20 that receives said primary bag 10, as shown in FIGS. 1 and 2. Optionally, as shown in FIG. 3, it is possible to provide a tertiary bag 30 that receives said secondary bag 20.

Air may be evacuated either from between the primary bag 10 and the secondary bag 20 for a two-bag configuration, or, where appropriate, from between the secondary bag 20 and the tertiary bag 30 for a three-bag configuration. The vacuum makes it possible to detect any possible problem of integrity that would appear as a loss of vacuum causing the said bags to become separated.

FIGS. 1 and 2 show a two-bag configuration.

In the embodiment in FIG. 1, the primary bag 10 is sealed, while in the embodiment in FIG. 2, it includes a gate 40 that is suitable for co-operating with an appropriate bag reception device so as to transfer the contents of the primary bag 10 directly into a chamber having a controlled atmosphere.

In these two-bag configurations, the primary bag 10 is made out of a material having good impermeability to air, such as polyethylene or aluminum, for example. Other equivalent materials may also be envisaged.

The secondary bag 20 may then advantageously be a three-layer polyethylene/polyamide/polyethylene (PE/PA/PE) bag.

Other materials may be envisaged providing they make it possible to establish and to maintain a vacuum between the primary bag 10 and the secondary bag 20.

In the embodiment in FIG. 3, when there is a tertiary bag 30, the primary bag 10 is made out of a material that is permeable to air, such as polyethylene, in particular Tyvek®, for example. Other equivalent materials may also be envisaged.

In this configuration, the secondary bag 20 may also include a plurality of layers, in particular a four-layer structure with an inner layer made of polyethylene terephthalate, a layer made of aluminum, a layer made of polyamide, and an outer layer made of polyethylene.

The tertiary bag 30 may also be multilayered, e.g. with an inner layer made of polyethylene, a central layer made of polyamide, and an outer layer made of polyethylene.

Other materials may be envisaged for these secondary and tertiary bags 20, 30 providing they make it possible to establish and to maintain a vacuum between the secondary bag 20 and the tertiary bag 30.

In the invention, the atmosphere inside said primary bag 10 is mainly nitrogen based, with at least 80% nitrogen, advantageously at least 90% nitrogen.

Advantageously, this atmosphere is constituted substantially of nitrogen and of oxygen.

Optionally, said atmosphere may contain only nitrogen, i.e. 100% nitrogen.

For a two-bag configuration as shown in FIG. 1 or FIG. 2, the method of inserting nitrogen may, in particular, comprise the following steps:

filling the primary bag 10 with the parts 1 in the ambient atmosphere;

putting the filled primary bag 10 into place in an evacuation chamber so as to evacuate any oxygen contained inside said primary bag 10; and

inserting nitrogen into said primary bag 10, then sealing said primary bag 10.

Evacuation, followed by inserting nitrogen, may be monitored by means of pressure gauges.

For a three-bag configuration as shown in FIG. 3, the method of inserting nitrogen may, in particular, comprise the following steps:

filling the primary bag 10 that is permeable to air with the parts 1 in the ambient atmosphere;

closing said filled primary bag 10 that is permeable to air;

inserting the closed primary bag 10 into said secondary bag 20;

putting said secondary bag 20 into place in an evacuation chamber so as to evacuate any oxygen contained inside the primary and secondary bags 10, 20; and

inserting nitrogen into said secondary bag 20, then sealing said secondary bag 20.

Evacuation, followed by inserting nitrogen, may be monitored by means of pressure gauges.

### Sticking Test:

The table below shows the improved performance obtained with the present invention. The tested configuration was a two-bag configuration, in accordance with the embodiment of FIG. 1 or FIG. 2.

| | | Non-irradiated | | | |
|---|---|---|---|---|---|
| | | components | | Irradiated components | |
| | | | Packaging | | Packaging |
| | | Conventional | under | Conventional | under |
| Aging | Batch | packaging | nitrogen | packaging | nitrogen |
| T0 | A | 0/200 | 0/200 | 0/200 | 0/200 |
| | B | 0/200 | 0/200 | 0/200 | 0/200 |
| | C | 0/200 | 0/200 | 0/200 | 0/200 |

| | | | |
|---|---|---|---|
| T6 | A | 11/200 | 0/200 |
| | B | 3/200 | 0/200 |
| | C | 6/200 | 0/200 |
| T24 | A | 2/200 | 0/200 |
| | B | 5/200 | 0/200 |
| | C | 4/200 | 0/200 |

Each of the tested batches A, B, and C contained 1150 parts, with the results given being adjusted to be proportional to 200 parts. Each batch was tested firstly with a standard atmosphere (ambient air), and secondly with a nitrogen-based atmosphere, specifically about 100% nitrogen for the tests at the moment of packaging, both before and after gamma sterilization. In the tests, sterilization was performed with the parts receiving a dose of radiation of 35 kilograys (kGy), it being understood that normal doses are generally in the range 18 kGy to 32 kGy. The tests, consisting in counting the stuck-together parts in each batch, were performed at different time intervals, namely at T0 (after packaging) before and after sterilization, then at T6 (after six months of storage), and at T24 (after twenty-four months of storage).

It should be observed that in a prior-art packaging device in which the atmosphere contained in the primary bag 10 was a standard atmosphere formed of ambient air, the results after sterilization were always above the acceptable limit of 2/200 stuck-together parts, both at T6 and at T24.

In contrast, with the present invention, there were no stuck-together parts, neither before sterilization, nor after.

FIGS. 4 and 5 show the results on graphs.

The invention is described above more particularly with reference to stoppers for pharmaceutical containers, but it could also apply to other types of elastomer parts, such as syringe pistons or syringe needle-guards, for example.

The present invention is described above with reference to several particular embodiments, but naturally it is not limited by said embodiments, but on the contrary, any useful modifications can be applied thereto by the person skilled in the art, without going beyond the ambit of the present invention, as defined by the accompanying claims.

1. A packaging method for packaging elastomer parts (1), such as stoppers for pharmaceutical containers, the method comprising the following steps:
packaging parts (1) in a primary bag (10) made of material that is substantially impermeable to air; and
applying in said primary bag (10), a nitrogen-based atmosphere, with at least 80% nitrogen;
the method being characterized in that said primary bag (10) is packaged in a secondary bag (20), with a vacuum being applied between said primary bag (10) and said secondary bag (20).

2. A method according to claim 1, wherein the atmosphere applied in said primary bag (10) contains at least 90% nitrogen.

3. A method according to claim 2, wherein the atmosphere applied in said primary bag (10) contains 100% nitrogen.

4. A method according to claim 1, wherein the atmosphere applied in said primary bag (10) is constituted substantially of nitrogen and of oxygen.

5. A method according to claim 1, wherein said primary bag (10) that is substantially impermeable to air comprises polyethylene or aluminum.

6. A method according to claim 1, wherein said secondary bag (20) comprises a plurality of layers, in particular a three-layer polyethylene/polyamide/polyethylene (PE/PA/PE) structure.

7. A packaging method for packaging elastomer parts (1), such as stoppers for pharmaceutical containers, the method comprising the following steps:
packaging parts (1) in a primary bag (10) made of material that is permeable to air;
packaging said primary bag (10) in a secondary bag (20) made of material that is substantially impermeable to air; and
applying in said secondary bag (20), a nitrogen-based atmosphere, with at least 80% nitrogen;
the method being characterized in that said secondary bag (20) is packaged in a tertiary bag (30), with a vacuum being applied between said secondary bag (20) and said tertiary bag (30).

8. A method according to claim 7, wherein the atmosphere applied in said secondary bag (20) contains at least 90% nitrogen.

9. A method according to claim 8, wherein the atmosphere applied in said secondary bag (20) contains 100% nitrogen.

10. A method according to claim 7, wherein the atmosphere applied in said secondary bag (20) is constituted substantially of nitrogen and of oxygen.

11. A method according to claim 7, wherein said primary bag (10) that is permeable to air comprises polyethylene, in particular Tyvek®.

12. A method according to claim 7, wherein said secondary bag (20) includes a plurality of layers, in particular a four-layer structure with an inner layer made of polyethylene terephthalate, a layer made of aluminum, a layer made of polyamide, and an outer layer made of polyethylene.

13. A method according to claim 7, wherein said tertiary bag (30) comprises polyethylene.

14. A method according to claim 1, wherein, after applying the nitrogen-based atmosphere in the primary bag (10) or in the secondary bag (20), said parts (1) are subjected to gamma sterilization.

15. A packaging device for packaging elastomer parts (1), such as stoppers for pharmaceutical containers, the device comprising a primary bag (10), made of material that is substantially impermeable to air, receiving said parts (1), said primary bag (10) containing a nitrogen-based atmosphere, with at least 80% nitrogen, the device being characterized in that said primary bag (10) is packaged in a secondary bag (20), with a vacuum being applied between said primary bag (10) and said secondary bag (20).

16. A device according to claim 15, wherein the atmosphere in said primary bag (10) contains at least 90% nitrogen.

17. A device according to claim 16, wherein the atmosphere in said primary bag (10) contains 100% nitrogen.

18. A device according to claim 15, wherein the atmosphere in said primary bag (10) is constituted substantially of nitrogen and of oxygen.

19. A device according to claim 15, wherein said primary bag (10) comprises polyethylene or aluminum.

20. A device according to claim 15, wherein said secondary bag (20) comprises a plurality of layers, in particular a three-layer polyethylene/polyamide/polyethylene (PE/PA/PE) structure.

21. A packaging device for packaging elastomer parts (1), such as stoppers for pharmaceutical containers, the device comprising a primary bag (10), made of material that is permeable to air, receiving said parts (1), said primary bag (10) being packaged in a secondary bag (20) made of material that is substantially impermeable to air, said primary bag (10) and said secondary bag (20) containing a nitrogen-based atmosphere, with at least 80% nitrogen, the device being characterized in that said secondary bag (20) is packaged in a tertiary bag (30), with a vacuum being applied between said secondary bag (20) and said tertiary bag (30).

22. A device according to claim 21, wherein the atmosphere in said primary and secondary bags (10, 20) contains at least 90% nitrogen.

23. A device according to claim 22, wherein the atmosphere in said primary and secondary bags (10, 20) contains 100% nitrogen.

24. A device according to claim 21, wherein the atmosphere in said primary and secondary bags (10, 20) is constituted substantially of nitrogen and of oxygen.

25. A device according to claim 21, wherein said primary bag (10) that is permeable to air comprises polyethylene, in particular Tyvek®.

26. A device according to claim 21, wherein said secondary bag (20) includes a plurality of layers, in particular a four-layer structure with an inner layer made of polyethylene terephthalate, a layer made of aluminum, a layer made of polyamide, and an outer layer made of polyethylene.

27. A device according to claim 21, wherein said tertiary bag (30) comprises polyethylene.
